(19) United States
(12) Patent Application Publication
(10) Pub. No.: US 2008/0091103 A1
(43) Pub. Date: Apr. 17, 2008

(54) System For And Method Of Guiding An Object

(76) Inventors: Satish Sundar, Milpitas, CA (US); Bruno Strul, Portola Valley, CA (US)

Correspondence Address: PERMAN & GREEN, 425 POST ROAD, FAIRFIELD, CT, 06824, US

(21) Appl. No.: 10/568,163

(22) PCT Filed: Aug. 9, 2004
(86) PCT No.: PCT/GB04/03453

§ 371 Date: Aug. 2, 2007

Related U.S. Application Data
(60) Provisional application No. 60/493,602, filed on Aug. 9, 2003.
(60) Provisional application No. 60/496,259, filed on Aug. 19, 2003.

(51) Int. Cl. A61B 8/12

(52) U.S. Cl. 600/439; 600/459; 600/461

(57) ABSTRACT

A system for and a method of guiding an object, in particular an elongate object, such as a needle, into a region of interest, such as a body cavity, which is reached by maneuvering between hard structures, such as bone structures, for example, two adjacent vertebrae in epidural anesthesia.

The present invention relates to a system for and a method of guiding an object, in particular an elongate object, such as a needle, into a region of interest, such as a body cavity, which is reached by maneuvering between hard structures, such as bone structures, for example, two adjacent vertebrae in epidural anesthesia.

Imaging techniques, such as ultrasonic imaging [1], X-ray fluorescence imaging and magnetic resonance imaging (MRI), have been used for some time to image body structures, particularly in human structures.

It is an aim of the present invention to utilize aspects of such imaging technologies, albeit in a highly-modified form, such as to enable the guiding of an object, in particular an elongate object, such as a needle, into a region of interest, such as a body cavity, which is reached by maneuvering between hard structures, such as bone structures, for example, two adjacent vertebrae in epidural anesthesia.

In one aspect the present invention provides a system for use in guiding an object, in particular an elongate object, into a region of interest which is reached by maneuvering between structures, the system comprising: at least one emitter array for emitting an output; at least one receiver array for receiving an input; a drive unit operably connected to the at least one emitter array to drive the same to emit an output; an acquisition unit operably connected to the at least one receiver array to acquire an input as received thereby; a modeling unit for modeling the input as acquired by the acquisition unit to identify structures as obstacles to guiding of the object; a path determination unit for determining an obstacle-free path between the identified obstacle structures; and an indicator unit for indicating to an operator whether the object is being guided in accordance with the obstacle-free path.

In another aspect the present invention provides a system for guiding an object, in particular an elongate object, into a region of interest which is reached by maneuvering between structures, the system comprising: at least one emitter array for emitting an output; at least one receiver array for receiving an input; a drive unit operably connected to the at least one emitter array to drive the same to emit an output; an acquisition unit operably connected to the at least one receiver array to acquire an input as received thereby; a modeling unit for modeling the input as acquired by the acquisition unit to identify structures as obstacles to guiding of the object; and a path determination unit for determining an obstacle-free path between the identified obstacle structures in accordance with which the object is in use guided.

In a further aspect the present invention provides a transducer element comprising a body element which is to be located on a surface of a body, a projecting element which extends from the body element and is configured in use to pierce the body and be located therewithin, and at least one transducer array which is disposed in the projecting element.

In a yet further aspect the present invention provides a coupling assembly for movably coupling a device, which includes a body and an elongate object extending therefrom, to a mounting pad, the coupling assembly comprising a connector to which the body of the device is connected and a flexible attachment member which connects the connector to the mounting pad such as to allow for movement of the device relative to the mounting pad.

In a still further aspect the present invention provides a transducer assembly for attachment to a surface of a body, the transducer assembly comprising a mounting pad which includes an aperture through which an object is insertable, and at least one transducer array.

In yet another aspect the present invention provides an elongate element for insertion into a body, the element incorporating at least one transducer array.

In still yet another aspect the present invention provides an elongate element for insertion into a body, the element incorporating at least one transponder.

In a still further aspect the present invention provides a method for use in guiding an object, in particular an elongate object, into a region of interest which is reached by maneuvering between structures, the method comprising the steps of: emitting an output to the region of interest; receiving an input from the region of interest; acquiring the input as received; modeling the acquired input to identify structures as obstacles to guiding of the object; determining an obstacle-free path between the identified obstacle structures; and indicating to an operator whether the object is being guided in accordance with the obstacle-free path.

In yet still another aspect the present invention provides a method of guiding an object, in particular an elongate object, into a region of interest which is reached by maneuvering between structures, the method comprising the steps of: emitting an output to the region of interest; receiving an input from the region of interest; acquiring the input as received; modeling the acquired input to identify structures as obstacles to guiding of the object; determining an obstacle-free path between the identified obstacle structures; and guiding the object in accordance with the obstacle-free path.

Preferred embodiments of the present invention will now be described hereinbelow by way of example only with reference to the accompanying drawings, in which:

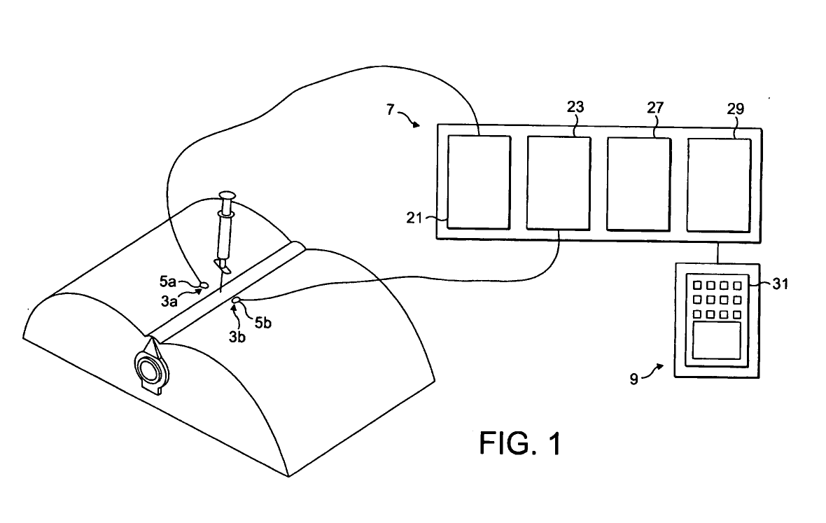

FIG. 1 illustrates an object-guiding system in accordance with a first embodiment of the present invention, where embodied in inserting the needle of a delivery device between vertebrae of the spinal column of a human subject;

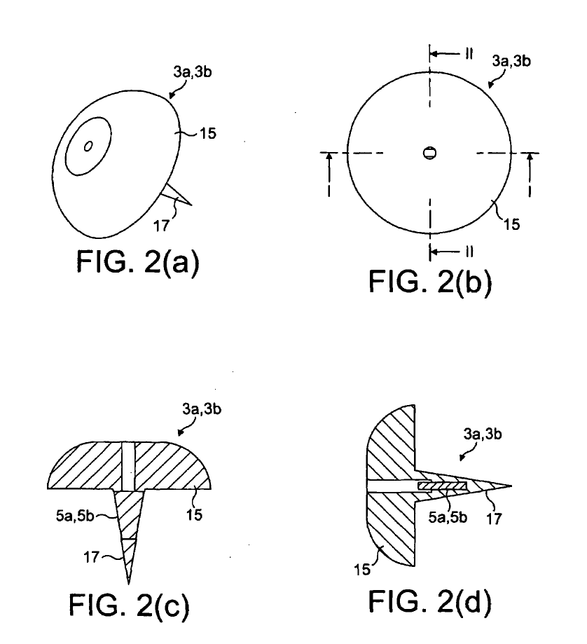

FIG. 2(a) illustrates a perspective view of a transducer element of the system of FIG. 1;

FIG. 2(b) illustrates a plan view of the transducer element of FIG. 2(a);

FIG. 2(c) illustrates a first vertical sectional view (along section I-I in FIG. 2(b)) of the transducer element of FIG. 2(a);

FIG. 2(d) illustrates a second vertical sectional view (along section II-II in FIG. 2(b)) of the transducer element of FIG. 2(a);

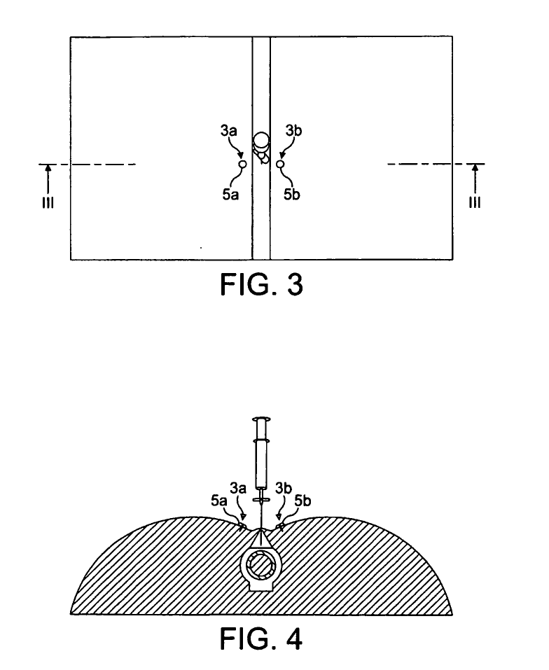

FIG. 3 illustrates a plan view of the back of a subject showing the attachment of the transducer elements adjacent the spinal column of the subject;

FIG. 4 illustrates a vertical sectional view (along section III-III) through the back of the subject as illustrated in FIG. 3;

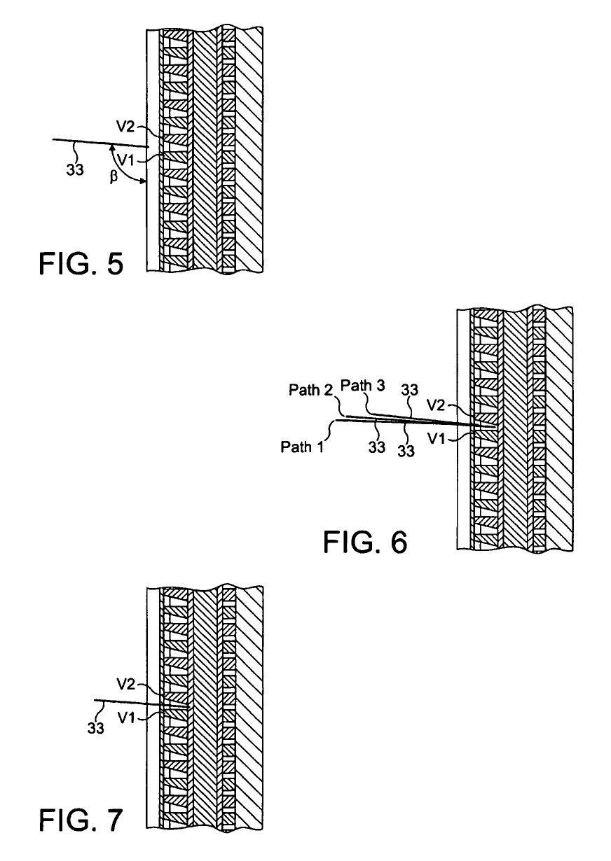

FIG. 5 illustrates the location of a needle at the surface of the skin of a subject at the commencement of insertion of the needle between adjacent vertebrae;

FIG. 6 illustrates the re-alignment of a needle during insertion of the needle between adjacent vertebrae;

FIG. 7 illustrates a needle where fully inserted to the epidural cavity between adjacent vertebrae;

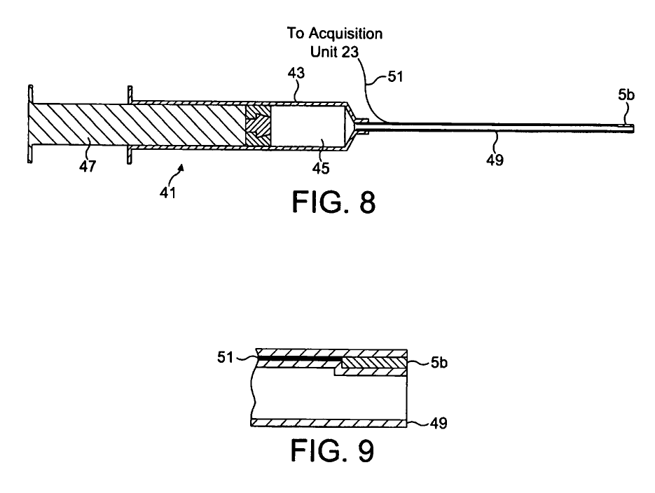

FIG. 8 illustrates a longitudinal sectional view through a delivery device of an object-guiding system in accordance with a second embodiment of the present invention;

FIG. 9 illustrates an enlarged sectional view of the tip of the needle of the delivery device of FIG. 8;

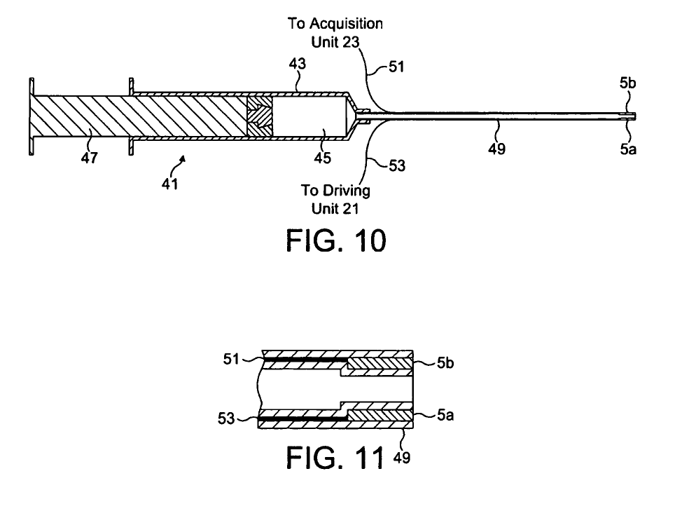

FIG. 10 illustrates a longitudinal sectional view through a delivery device of an object-guiding system in accordance with a third embodiment of the present invention;

FIG. 11 illustrates an enlarged sectional view of the tip of the needle of the delivery device of FIG. 10;

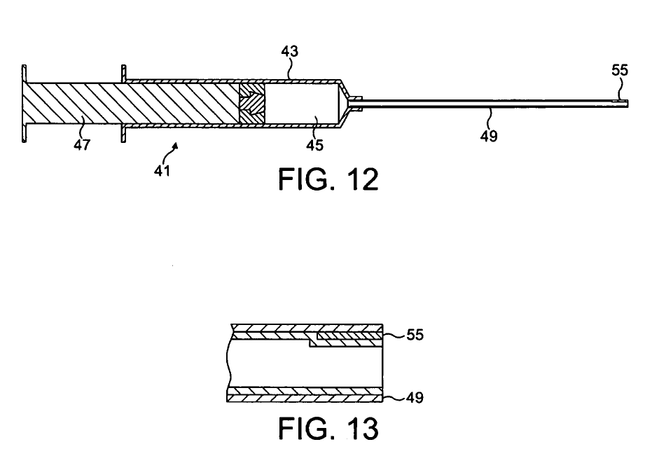

FIG. 12 illustrates a longitudinal sectional view through a delivery device of an object-guiding system in accordance with a fourth embodiment of the present invention;

FIG. 13 illustrates an enlarged sectional view of the tip of the needle of the delivery device of FIG. 12;

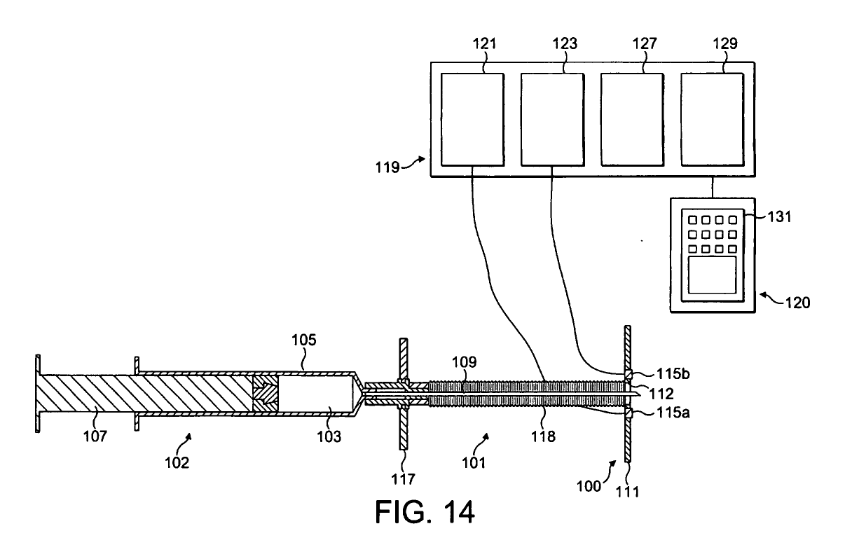

FIG. 14 illustrates an object-guiding system in accordance with a fifth embodiment of the present invention;

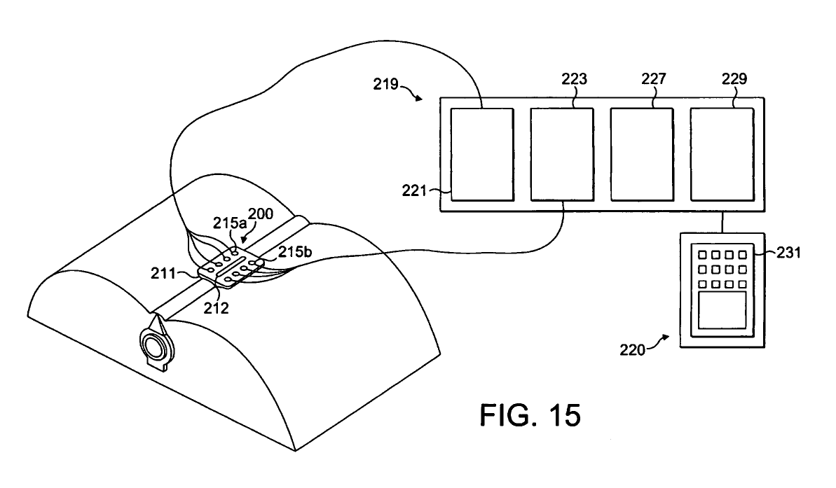

FIG. 15 illustrates an object-guiding system in accordance with a sixth embodiment of the present invention, where embodied for inserting the needle of a delivery device between vertebrae of the spinal column of a human subject;

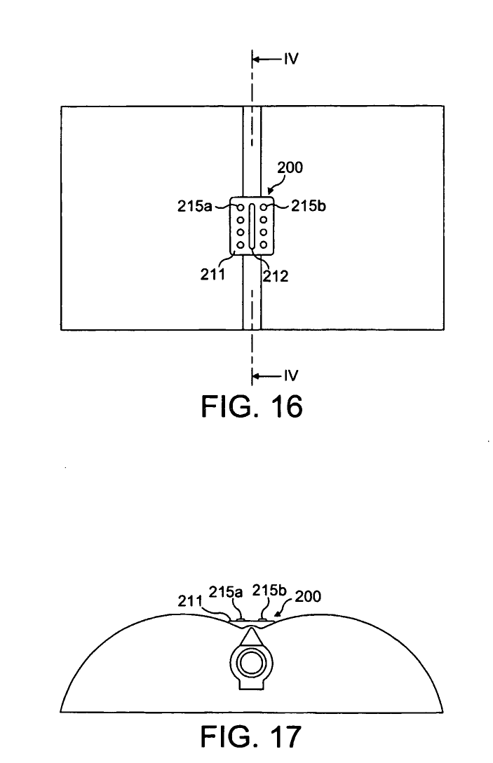

FIG. 16 illustrates a plan view of the back of the subject showing the attachment of the transducer assembly adjacent the spinal column of the subject;

FIG. 17 illustrates an end elevational view of the back of the subject showing the attachment of the transducer assembly adjacent the spinal column of the subject; and

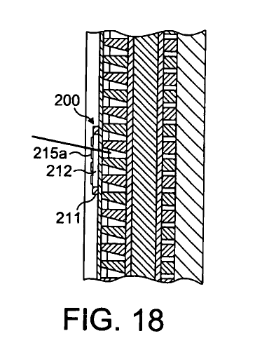

FIG. 18 illustrates a longitudinal sectional view (along section IV-IV in FIG. 16) through the spinal column of the subject.

FIGS. 1 and 2 illustrate an object-guiding system in accordance with a first embodiment of the present invention.

The system comprises at least one transducer element 3, which comprises at least one emitter array 5a and at least one receiver array 5b, in this embodiment ultrasonic transducer arrays, a control unit 7 which is operably connected to the emitter and receiver arrays 5a, 5b to image bone structures between which an object, in this embodiment a needle, is to be inserted and the needle during insertion therebetween and determine therefrom the insertion path, and an indicator unit 9 for indicating information to the operator concerning the insertion path of the needle, such as to enable the user to receive feedback concerning the insertion path of the needle and correct, as necessary, the insertion path of the needle during insertion. In one embodiment the control and indicator units 7, 9 can be provided as an integral unit.

In this embodiment the system comprises first and second transducer elements 3a, 3b which each house a respective one of the emitter array 5a and the receiver array 5b. In an alternative embodiment the system could comprise a single transducer element 3 which houses both the emitter array 5a and the receiver array 5b.

In this embodiment the transducer elements 3a, 3b have the form of thumbtacks, typically formed of a plastics material, which each comprise a body 15 which is located on the surface of the skin of the subject, and a sharp projection 17 which extends from the body 15 and in use penetrates into the fleshy tissue beneath the skin of the subject proximate the bone structures of interest. In this embodiment the projection 17 houses the respective transducer array 5a, 5b, such that the transducer arrays 5a, 5b can be located close to the bone structures of interest, thereby improving the imaging. By providing the transducer elements 3a, 3b as thumbtacks, the fixing of the transducer elements 3a, 3b is facilitated, in not necessarily requiring the use of any special glues or gels.

In an alternative embodiment the transducer elements 3a, 3b, again typically formed of a plastics material, could each only comprise a body 15 which houses the respective transducer array 5a, 5b, with the body 15 being adhered to the skin by an adhesive tape or glue, and optionally with the use of a gel to prevent the occurrence of air bubbles at the interface of the body 15 and the surface of the skin.

The control unit 7 includes a drive unit 21 for driving the at least one emitter array 5a to emit an output, in this embodiment an ultrasonic output, and an acquisition unit 23 which receives an input, in this embodiment an ultrasonic input, from the at least one receiver array 5b. In this embodiment the drive unit 21 and the acquisition unit 23 have a wired connection to the emitter and receiver arrays 5a,5b, but in an alternative embodiment the connections could be wireless. As will be appreciated, wireless communication allows for remote operation of the system, thereby allowing for tele-operation of the system from a remote location thousands of miles away from the subject.

In this embodiment the drive unit 21 is driven such as to drive the at least one emitter array 5a to emit an output of varying phase, which provides that the input as received by the at least one receiver array 5b represents an image which shows the different densities of the imaged materials.

In this embodiment the acquisition unit 23 stores the input as received from the at least one receiver array 5b as a three-dimensional grid and assigns sections of the grid having densities within predetermined ranges of density as corresponding to respective kinds of materials, such as bone, ligament layers, fat layers and the like, and also the needle. The resolution of the grid, that is, the coarseness/fineness of the grid, can be varied by varying the sensitivity, number and arrangement of the transducer arrays 5a, 5b.

The control unit 7 further includes a modeling unit 27 which repeatedly models the data as assigned by the acquisition unit 23 to open or closed surfaces, each enclosing a volume of the same or similar density, representing bone, ligament layers, fat layers and the like and also the needle, using appropriate surface-generating approaches, such as B-patches and the like [2]. For epidural anesthesia, the bone structures are adjacent vertebrae, and the needle is required to be accurately inserted between the adjacent vertebrae to reach the epidural cavity. In one embodiment the re-modeling need only be partial, insofar as the position of only certain proximate surfaces is required, as will become apparent hereinbelow.

The control unit 7 further includes a path determination unit 29 which is operative repeatedly to determine a curve, hereinafter referred to as an obstacle-free curve, which defines an insertion path for a needle between the modeled bone structures which maintains sufficient clearance from the bone structures during insertion of the needle. This determination is computed based on the modeled bone structures and the current position of the needle as modeled by the modeling unit 27. In this embodiment computation is implemented in hard real time, but could be implemented in soft real time. Such methods of computation are well known in the field of obstacle-avoidance and the field of robotics, in particular mobile robot navigation [3-6].

For many cases, the obstacle-free curve will be a straight line. In some cases, however, it may not be possible to move the needle along a straight line of travel. Reasons for this can include the operator not starting the insertion at a location or on a path which allows for a straight-line insertion, which can be intentional, the geometry of the vertebrae not permitting a straight-line insertion, and the subject moving during insertion in such a way as to prevent a straight-line insertion.

The indicator unit 9 comprises an indicator 31 which indicates whether the current insertion path of the needle is correct, that is, within a window as defined by the obstacle-free curve as determined by the path determination unit 29. In one embodiment the indicator 31 may be mounted to the delivery device, so as to avoid the operator having to watch other than the delivery device.

In this embodiment the indicator 31 comprises a visual indicator, here LEDs which are illuminated in a color dependent upon the correctness of the current insertion path of the needle, that is, in terms of both the angle of approach and the insertion location. For example, flashing green lights may be used to signify a good current angle of approach and/or insertion location, flashing yellow lights may be used to warn that the current angle of approach and/or insertion location is close to being outside the window as defined by the obstacle-fee curve, and flashing red lights may be used to indicate that the current angle of approach and/or insertion location is incorrect, that is, outside the window as defined by the obstacle-free curve.

In another embodiment the indicator 31 could alternatively or additionally comprise an audible indicator. For example, an intermittent tone with a first period may be used to signify a good current angle of approach and/or insertion location, an intermittent tone with a progressively shorter period may be used to warn that the current angle of approach and/or insertion location are close to being outside the window as defined by the obstacle-fee curve, with the period becoming shorter in relation to the closeness to the edge of the window, and a continuous tone may be used to indicate that the current angle of approach and/or insertion location is incorrect.

In a further embodiment the indicator 31 could comprise a display which displays a modeled image of the needle during insertion between adjacent bone structures, providing the operator with a clear graphic representation of the path to be followed.

Operation of the object-guiding system will now be described hereinbelow with reference to FIGS. 3 to 6 of the accompanying drawings.

An operator attaches the transducer elements 3a, 3b to the body of the subject, in this embodiment to the respective sides of the vertebrae V1, V2 between which a needle 33 is to be inserted for epidural delivery, as illustrated in FIGS. 3 and 4. In this embodiment the sharp projections 17 of the transducer elements 3a, 3b are pressed into the skin of the subject until the bodies 15 of the transducer elements 3a, 3b abut the surface of the skin of the subject. As necessary, local anesthetic can be used to numb the puncture sites, and a needle can be used to pre-puncture the surface of the skin.

The operator then actuates the system to model the vertebrae V1, V2 and the needle 33, where present, using the modeling unit 27 and repeatedly determine the obstacle-free curve using the determination unit 29.

Following actuation of the system, the operator then locates the tip of the needle 33 at the surface of the skin and begins to insert the needle 33 into the skin, as illustrated in FIG. 5, with an angle of approach β.

In this embodiment, where the location of the tip of the needle 33 is correct, the indicator 31 affirms the correct position by illuminating the LEDs as a flashing green light, but, where the location of the tip of the needle 33 is close to the edge of the window as defined by the obstacle-free path, the indicator 31 indicates the closeness to the edge of the window by illuminating the LEDs as a flashing yellow light, and, where the location of the tip of the needle 33 is outside the window as defined by the obstacle-free path, the indicator 31 indicates this incorrect positioning by illuminating the LEDs as a flashing red light and the operator would re-position the needle 33 until the needle was located within the window as defined by the obstacle free path, this being represented by the LEDs of the indicator 31 being illuminated as one of a flashing green or yellow light.

The operator then continues to insert the needle 33 between the vertebrae V1, V2.

Where the angle of approach β of the needle 33 remains correct and falls well within the window as defined by the obstacle-free path, the indicator 31 affirms the correct angle of approach β by illuminating the LEDs as a flashing green light, and the operator continues to insert the needle 33.

Where the angle of approach β of the needle 33 is such that the needle 33 is close to the edge of the window as defined by the obstacle-free path, the indicator 31 indicates the closeness to the edge of the window by illuminating the LEDs as a flashing yellow light, and the operator is required to be alert to the position of the needle 33 in continuing to insert the needle 33.

Where the angle of approach β of the needle 33 falls outside the window as defined by the obstacle-free path, the indicator 31 indicates this incorrect positioning by illuminating the LEDs as a flashing red light, requiring the operator to correct the angle of approach β of the needle 33 until the needle 33 is located within the window as defined by the obstacle-free path before continuing further to insert the needle 33, this being represented by the LEDs of the indicator 31 being illuminated as one of a flashing green or amber light. This correction of the angle of approach β of the needle 33 is repeated any number of times. By way of exemplification, FIG. 6 represents the correction of the angle of approach β of the needle 33 from Path A, through Path B and finally to Path C which allows for complete insertion between the vertebrae V1, V2 as required. In correcting the angle of approach β of the needle 33, the operator can change angle of approach β of the needle 33 in discrete steps or continuously, which in effect corresponds to a large number of very small discrete steps.

Following this procedure, insertion of the needle 33 continues until the needle 33 is inserted into the required cavity, in this embodiment the epidural cavity, as illustrated in FIG. 7.

FIGS. 8 and 9 illustrate the delivery device 41 of an object-guiding system in accordance with a second embodiment of the present invention.

The system of this embodiment is similar to the system of the above-described first embodiment, and thus, in order to avoid unnecessary duplication of description only the differences will be described in detail, with like parts being designated by like reference signs.

The system of this embodiment differs from that of the above-described first embodiment in comprising a modified delivery device 41.

The delivery device 41 comprises a body 43 which defines a fluid chamber 45, a plunger 47 which is slideably disposed in the fluid chamber 45 such as to be operable to expel fluid therefrom, and an elongate tubular element 49, in this embodiment a needle, which is fluidly connected to the fluid chamber 45. Embodiments of such a delivery device are disclosed in the applicant's earlier PCT/GB2004/00338, the content of which is incorporated herein by reference.

In this embodiment the at least one receiver array 5b is disposed to the needle 49 of the delivery device 41, in this embodiment the distal end of the needle 49, instead of being housed in a separate transducer element 3b, with a connecting lead 51 passing along and out of the needle 49 for connection to the acquisition unit 23.

In this embodiment the needle 49 is a laminated structure, with the connecting lead 51 passing along the needle 49 between laminates.

In an alternative embodiment the at least one emitter array 5a could instead be disposed to the needle 49.

Operation of this system is the same as for the system of the above-described first embodiment.

FIGS. 10 and 11 illustrate the delivery device 41 of an object-guiding system in accordance with a third embodiment of the present invention.

The system of this embodiment is very similar to the system of the above-described second embodiment, and thus, in order to avoid unnecessary duplication of description only the differences will be described in detail, with like parts being designated by like reference signs.

The system of this embodiment differs from that of the above-described second embodiment in that both the emitter and receiver arrays 5a, 5b are disposed to the needle 49 of the delivery device 41, in this embodiment the distal end of the needle 49, instead of being housed in a separate transducer elements 3a, 3b, with connecting leads 51, 53 passing along and out of the needle 49 for connection to respective ones of the driving and acquisition units 21, 23.

FIGS. 12 and 13 illustrate the delivery device 41 of an object-guiding system in accordance with a fourth embodiment of the present invention.

The system of this embodiment is very similar to the system of the above-described first embodiment, and thus, in order to avoid unnecessary duplication of description only the differences will be described in detail, with like parts being designated by like reference signs.

The system of this embodiment differs from that of the above-described first embodiment in comprising a modified delivery device 41.

The delivery device 41 comprises a body 43 which defines a fluid chamber 45, a plunger 47 which is slideably disposed in the fluid chamber 45 such as to be operable to expel fluid therefrom, and an elongate tubular element 49, in this embodiment a needle, which is fluidly connected to the fluid chamber 45. Embodiments of such a delivery device are disclosed in the applicant's earlier PCT/GB2004/00338, the content of which is incorporated herein by reference.

In this embodiment the needle 49 of the delivery device 41, in this embodiment the distal end thereof, includes a transponder 55 which is actuated by the output of the at least one emitter array 5a to generate a signature which is readily identifiable in the input of the acquisition unit 23, thus enabling the position of the tip of the needle 49 to be accurately determined without necessarily having to model the image as acquired by the acquisition unit 23 to identify the needle 49 to determine the position of the tip thereof.

Operation of the system of this embodiment is otherwise the same as for the system of the above-described first embodiment.

FIG. 14 illustrates an object-guiding system in accordance with a fifth embodiment of the present invention.

The system comprises a transducer assembly 100 which is attached to the skin of a subject, and a coupling assembly 101 for movably coupling a delivery device 102 to the transducer assembly 100 such as to facilitate controlled movement of the delivery device 102 relative to the transducer assembly 100.

The delivery device 102 comprises a body 103 which defines a fluid chamber 105, a plunger 107 which is slideably disposed in the fluid chamber 105 such as to be operable to expel fluid therefrom, and an elongate tubular element 109, in this embodiment a needle, which is fluidly connected to the fluid chamber 105. Embodiments of such a delivery device are disclosed in the applicant's earlier PCT/GB2004/00338, the content of which is incorporated herein by reference.

The transducer assembly 100 comprises a mounting pad 111 which is attached to the skin of a subject and includes an aperture 112 through which the needle 109 of the delivery device 102 is insertable, and at least one transducer array 115, in this embodiment at least one emitter array 115a and at least one receiver array 115b, in this embodiment ultrasonic transducer arrays, which are disposed concentrically with the aperture 112 in the mounting pad 111.

In this embodiment the mounting pad 111 is formed of a flexible polymeric material, such as a polycarbonate, which ensures a close air-free interface between the skin and the transducer arrays 115a, 115b. Gels, suitable glues and adhesive tapes can also be used to promote a good interfacial contact between the transducer arrays 115a, 115b and the surface of the skin of the subject.

The coupling assembly 101 comprises a connector 117 which is fixedly connected to the body 103 of the delivery device 102, and a flexible attachment member 118 which connects the connector 117 to the mounting pad 111, such as to allow for movement of the delivery device 102, and hence the needle 109 thereof, both axially and laterally relative to the mounting pad 111.

In this embodiment the attachment member 118 comprises a bellows structure, typically formed of a metal, such as stainless steel, or a plastics material, such as polycarbonate, which can be compressed axially to allow for insertion of the needle 109 of the delivery device 102 and also flexed laterally allowing the angle of approach of the needle 109 of the delivery device 102 to be altered while the mounting pad 111 remains fixed in position to the skin of the subject.

In an alternative embodiment the connector 117 could comprise a housing, typically formed of a metal, such as stainless steel, or a plastics material, such as polycarbonate, in which the body 103 of the delivery device 102 is slideable, such as to allow for axial movement of the body 103 of the delivery device 102, and hence the needle 109 thereof, relative to the mounting pad 111, and the flexible attachment member 118 could comprise a sheath which allows for lateral movement, such as to allow for the angle of approach of the needle 109 of the delivery device 102 to be altered while the mounting pad 111 remains fixed in position to the skin of the subject.

The system further comprises a control unit 119 which is operably connected to the emitter and receiver arrays 115a, 115b to image bone structures between which the needle 109 of the delivery device 102 is to be inserted and determine therefrom the insertion path, and an indicator unit 120 for indicating information to an operator concerning the insertion path of the needle 109, such as to enable the operator to receive feedback concerning the insertion path of the needle 109 and correct, as necessary, the insertion path of the needle 109 during insertion.

In an alternative embodiment one or both of the at least one emitter array 115a and the at least one receiver array 115b could be housed in elements separate from the mounting pad 103, for example, in thumbtack transducer elements of the kind as employed in the above-described first embodiment.

In yet another alternative embodiment the system could comprise a single transducer element which houses both the at least one emitter array 115a and the at least one receiver array 115b.

In an alternative embodiment, again as described hereinabove in relation to the first-described embodiment, the transducer arrays 115a, 115b could be housed in transducer elements which are adhered to the skin by an adhesive tape or glue, and optionally with the use of a gel to prevent the occurrence of air bubbles at the interface with the surface of the skin.

The control unit 119 includes a drive unit 121 for driving the at least one emitter array 115a to emit an output, in this embodiment an ultrasonic output, and an acquisition unit 123 which receives an input, in this embodiment an ultrasonic input, from the at least one receiver array 115b. In this embodiment the drive unit 121 and the acquisition unit 123 have a wired connection to the emitter and receiver arrays 115a, 115b, but in an alternative embodiment the connections could be wireless. As will be appreciated, wireless communication allows for remote operation of the system, thereby allowing for tele-operation of the system from a remote location thousands of miles away from the subject.

In this embodiment the drive unit 121 is driven such as to drive the at least one emitter array 115a to emit an output of varying phase, which provides that the input as received the at least one receiver array 115b represents an image which shows the different densities of the imaged materials.

In this embodiment the acquisition unit 123 stores the input as received from the at least one receiver array 115b as a three-dimensional grid and assigns sections of the grid having densities within predetermined ranges of density as corresponding to respective kinds of materials, such as bone, ligament layers, fat layers and the like, and also the needle. The resolution of the grid, that is, the coarseness/fineness of the grid, can be varied by varying the sensitivity, number and arrangement of the transducer arrays 115a, 115b.

The control unit 119 further includes a modeling unit 127 which is operative repeatedly to model the assigned data as acquired by the acquisition unit 123 to open or closed surfaces, each enclosing a volume of the same or similar density, representing bone, ligament layers, fat layers and the like and also the needle 109, using appropriate surface-generating approaches, such as B-patches and the like [2]. For epidural anesthesia, the bone structures are adjacent vertebrae, and the needle 109 is required to be accurately inserted between the adjacent vertebrae. In one embodiment the re-modeling need only be partial, insofar as the position of only certain proximate surfaces is required, as will become apparent hereinbelow.

The control unit 119 further includes a path determination unit 129 which is operative repeatedly to determine a curve, hereinafter referred to as an obstacle-free curve, which defines an insertion path for the needle 109 between the modeled bone structures which maintains sufficient clearance from the bone structures during insertion of the needle 109. This determination is computed based on the modeled bone structures and the current position of the needle 109 as modeled by the modeling unit 127. In this embodiment computation is implemented in hard real time, but could be implemented in soft real time. Such methods of computation are well known in the field of obstacle-avoidance and in the fields of robotics and mobile robot navigation [3-6].

For many cases, the obstacle-free curve will be a straight line. In some cases, however, it may not be possible to move the needle 109 along a straight line of travel. Reasons for this can include the operator not starting the insertion at a location or on a path which allows for a straight-line insertion, which can be intentional, the geometry of the vertebrae not permitting a straight-line insertion, and the subject moving during insertion in such a way as to prevent a straight-line insertion.

The indicator unit 120 comprises an indicator 131 which indicates whether the current insertion path of the needle 109 is correct, that is, within a window as defined by the obstacle-free curve as determined by the path determination unit 129. In one embodiment the indicator 131 may be mounted to the delivery device 102, so as to avoid the operator having to concentrate on other than the delivery device 102.

In this embodiment the indicator 131 comprises a visual indicator, here LEDs which are illuminated in a color dependent on the correctness of the current insertion path of the needle 109, that is, in terms of both the angle of approach and the insertion location. For example, flashing green lights may be used to signify a good current angle of approach and/or insertion location, flashing yellow lights may be used to warn that the current angle of approach and/or insertion location is close to being outside the window as defined by the obstacle-fee curve, and flashing red lights may be used to indicate that the current angle of approach and/or insertion location is incorrect, that is, outside the window as defined by the obstacle-free curve.

In another embodiment the indicator 131 could alternatively or additionally comprise an audible indicator. For example, an intermittent tone with a first period may be used to signify a good current angle of approach and/or insertion location, an intermittent tone with a progressively shorter period may be used to warn that the current angle of approach and/or insertion location is close to being outside the window as defined by the obstacle-fee curve, with the period becoming shorter in relation to the closeness to the edge of the window, and a continuous tone may be used to indicate that the current angle of approach and/or insertion location is incorrect.

In a further embodiment the indicator 131 could comprise a display which displays a modeled image of the needle 109 during insertion between adjacent bone structures, providing the operator with a clear graphic representation of the path to be followed.

Operation of the system of this embodiment is the same as for the above-described first embodiment, with the flexible attachment member 118 of the coupling assembly 101 flexing to accommodate axial and lateral movement of the delivery device 102, and hence the needle 109 thereof, relative to the mounting pad 111 of the transducer assembly 100.

FIG. 15 illustrates an object-guiding system in accordance with a sixth embodiment of the present invention.

The system comprises a transducer assembly 200 which comprises a mounting pad 211 which is attached to the skin of a subject and includes an aperture 212, in this embodiment an elongate slot, through which a needle of a delivery device is insertable, and a plurality of transducer arrays 215, in this embodiment a plurality of emitter arrays 215a and a plurality of receiver arrays 215b, in this embodiment ultrasonic transducer arrays, which are disposed to opposed sides of the aperture 212 in the mounting pad 211.

In this embodiment the mounting pad 211 is a rigid element, here formed of a polymeric material, such as polycarbonate, which is attached to the skin of the subject, here by an adhesive tape to effect a good interfacial contact between the transducer arrays 215a, 215b and the surface of the skin of the subject. In an alternative embodiment the mounting pad 211 could be glued to the skin of the subject. As necessary, gels could be used to promote a good interfacial contact between the transducer arrays 215a, 215b and the surface of the skin of the subject.

In an alternative embodiment the mounting pad 211 could be a semi-rigid element, such as a rubber block.

In another alternative embodiment the mounting pad 211 could be formed from a flexible material, such as a tape.

In an alternative embodiment the mounting pad 211 could be provided as two pad parts which each support a plurality of transducer arrays 215a, 215b.

In yet another embodiment the mounting pad 211 could be attached to the skin of the subject by the application of a vacuum.

The system further comprises a control unit 219 which is operably connected to the emitter and receiver arrays 215a, 215b to image bone structures between which a needle of a delivery device is to be inserted and determine therefrom the insertion path, and an indicator unit 220 for indicating information to an operator concerning the insertion path of the needle, such as to enable the operator to receive feedback concerning the insertion path of the needle and correct, as necessary, the insertion path of the needle during insertion.

In an alternative embodiment ones of the emitter arrays 215a and the receiver arrays 215b could be housed in elements separate from the mounting pad 211, for example, in thumbtack transducer elements of the kind as employed in the above-described first embodiment.

The control unit 219 includes a drive unit 221 for driving the emitter arrays 215a to emit an output, in this embodiment an ultrasonic output, and an acquisition unit 223 which receives an input, in this embodiment an ultrasonic input, from the receiver arrays 215b. In this embodiment the drive unit 221 and the acquisition unit 223 have a wired connection to the emitter and receiver arrays 215a, 215b, but in an alternative embodiment the connections could be wireless. As will be appreciated, wireless communication allows for remote operation of the system, thereby allowing for tele-operation of the system from a remote location thousands of miles away from the subject.

In this embodiment the drive unit 221 is driven such as to drive the emitter arrays 215a to emit an output of varying phase, which provides that the input as received by the receiver arrays 215b represents an image which shows the different densities of the imaged materials.

In this embodiment the acquisition unit 223 stores the input as received from the receiver arrays 215b as a three-dimensional grid and assigns sections of the grid having densities within predetermined ranges of density as corresponding to respective kinds of materials, such as bone, ligament layers, fat layers and the like, and also the needle. The resolution of the grid, that is, the coarseness/fineness of the grid, can be varied by varying the sensitivity, number and arrangement of the transducer arrays 215a, 215b.

The control unit 219 further includes a modeling unit 227 which is operative repeatedly to model the assigned data as acquired by the acquisition unit 223 to open or closed surfaces, each enclosing a volume of the same or similar density, representing bone, ligament layers, fat layers and the like and also the needle, using appropriate surface-generating approaches, such as B-patches and the like [2]. For epidural anesthesia, the bone structures are adjacent vertebrae, and the needle is required to be accurately inserted between the adjacent vertebrae. In one embodiment the re-modeling need only be partial, insofar as the position of only certain proximate surfaces is required, as will become apparent hereinbelow.

The control unit 219 further includes a path determination unit 229 which is operative repeatedly to determine a curve, hereinafter referred to as an obstacle-free curve, which defines an insertion path for the needle between the modeled bone structures which maintains sufficient clearance from the bone structures during insertion of the needle. This determination is computed based on the modeled bone structures and the current position of the needle as modeled by the modeling unit 227. In this embodiment computation is implemented in hard real time, but could be implemented in soft real time. Such methods of computation are well known in the field of obstacle-avoidance and in the fields of robotics and mobile robot navigation [3-6].

For many cases, the obstacle-free curve will be a straight line. In some cases, however, it may not be possible to move the needle along a straight line of travel. Reasons for this can include the operator not starting the insertion at a location or on a path which allows for a straight-line insertion, which can be intentional, the geometry of the vertebrae not permitting a straight-line insertion, and the subject moving during insertion in such a way as to prevent a straight-line insertion.

The indicator unit 220 comprises an indicator 231 which indicates whether the current insertion path of the needle is correct, that is, within a window as defined by the obstacle-free curve as determined by the path determination unit 229. In one embodiment the indicator 231 may be mounted to the delivery device, so as to avoid the operator having to concentrate on other than the delivery device.

In this embodiment the indicator 231 comprises a visual indicator, here LEDs which are illuminated in a color dependent on the correctness of the current insertion path of the needle, that is, in terms of both the angle of approach and the insertion location. For example, flashing green lights may be used to signify a good current angle of approach and/or insertion location, flashing yellow lights may be used to warn that the current angle of approach and/or insertion location is close to being outside the window as defined by the obstacle-fee curve, and flashing red lights may be used to indicate that the current angle of approach and/or insertion location is incorrect, that is, outside the window as defined by the obstacle-free curve.

In another embodiment the indicator 231 could alternatively or additionally comprise an audible indicator. For example, an intermittent tone with a first period may be used to signify a good current angle of approach and/or insertion location, an intermittent tone with a progressively shorter period may be used to warn that the current angle of approach and/or insertion location is close to being outside the window as defined by the obstacle-fee curve, with the period becoming shorter in relation to the closeness to the edge of the window, and a continuous tone may be used to indicate that the current angle of approach and/or insertion location is incorrect.

In a further embodiment the indicator 231 could comprise a display which displays a modeled image of the needle during insertion between adjacent bone structures, providing the operator with a clear graphic representation of the path to be followed.

Operation of the system of this embodiment is the same as for the above-described first embodiment, with the needle of the delivery device being inserted through the aperture 212 in the mounting pad 211. FIGS. 16 and 17 illustrate plan and end elevational views of the transducer assembly 200 as attached to the back of a subject above the spinal column, and FIG. 18 illustrates a longitudinal sectional view through the spinal column of the subject.

Finally, it will be understood that the present invention has been described in its preferred embodiments and can be modified in many different ways without departing from the scope of the invention as defined by the appended claims.

In one modification, the above-described systems could be modified to provide for automated insertion of the needle of the delivery device between the vertebrae. In this embodiment the position of the needle, both in terms of the insertion location and the angle of approach, may be controlled using a motor-controlled mechanism, such as a rack-and-pinion actuator, a lead screw actuator, a ball screw actuator, a solenoid actuator or a voice-coil actuator.

In the case of using a fully-automated system for advancing the needle of the delivery device, the role of the operator would be to monitor the obstacle-free curve, and manually make changes to that curve using the appropriate means of input. For example, he or she could change the control points of a B-spline representing the obstacle-free curve using, for example, a mouse, keystrokes or a light pen. Once the operator decides on the obstacle-free curve to be followed, the automated system is then used to move the needle along the specified path. The operator can interrupt or stop the travel of the needle at any time using a manual interlock, which could, for example, be actuated using a simple push button.

In another modification of the above-described systems, motor-controlled mechanisms, such as those described hereinabove, may be utilized as a means to advance the needle of the delivery device. In this case, the operator would have full control over the range of motion of the motor-controlled mechanisms, and could manually change the position of the mechanisms using a suitable means of input, such as a joystick, dial or the like. In this embodiment the needle would be advanced using input from the operator, much like when the operator manually advances the needle, using the obstacle-free curve as input.

In the above-mentioned configurations for automated advancement of the needle, pneumatic or hydraulic actuators could be used instead of motors or electromagnetic actuators.

It will also be appreciated that the present invention, although embodied in relation to the insertion of a needle between adjacent vertebrae for epidural anesthesia in the above-described embodiments, extends to the insertion of any elongate object through softer body materials, such as tissue, ligament or muscle, between hard structures, such as bone. For example, the present invention could be used in inserting a needle through the ribcage into the lungs.

It will be further appreciated that the present invention, although embodied in relation to the use of ultrasonic imaging, finds equal application with any other imaging technologies, such as X-ray fluoroscopy and magnetic resonance imaging (MRI), which allow for the identification of dense structures, particularly bone, and the object being inserted.

Further, for those embodiments where a fluid is to be delivered by the delivery device, in particular in an automated system, the delivery of fluid could be detected using a Doppler sensor.

### REFERENCES

[1] U.S. Pat. Nos. 6,572,554, 6,409,673, and 5,902,245.
[2] Michael E. Mortensen, Geometric Modeling, Second Edition, Wiley.
[3] E. Rimon and D. E. Koditschek, “Exact Robot Navigation Using Artificial Potential Functions”, IEEE Transactions on Robotics and Automation, Volume 8, Pages 501-518, 1992.
[4] Z. Shiller and S. Dubowsky, “Optimal Path Planning for Robotic Manipulators in the Presence of Obstacles, with Actuator, Gripper and Payload Constraints”, International Journal of Robotics Research, Volume 8, No 6, Pages 3-18, December 1989.
[5] M. Niv and D. M. Auslander, “Optimal Control of a Robot with Obstacles”, Proceedings of the 1984 American Control Conference, San Diego, Calif., Pages 280-287, June 1984.
[6] R. Volpe and P. Khosla, “Artificial Potentials with Elliptical Isopotential Contours for Obstacle Avoidance”, Proceedings of the IEEE International Conference on Robotics and Automation, 1987, Pages 180-185.

1. A system for use in guiding an object, in particular an elongate object, into a region of interest which is reached by maneuvering between structures, the system comprising:
at least one emitter array for emitting an output;
at least one receiver array for receiving an input;
a drive unit operably connected to the at least one emitter array to drive the same to emit an output;
an acquisition unit operably connected to the at least one receiver array to acquire an input as received thereby;
a modeling unit for modeling the input as acquired by the acquisition unit to identify structures as obstacles to guiding of the object;
a path determination unit for determining an obstacle-free path between the identified obstacle structures; and
an indicator unit for indicating to an operator whether the object is being guided in accordance with the obstacle-free path.

2. The system of claim 1, wherein the at least one emitter array is configured to emit an ultrasonic output and the at least one receiver array is configured to receive an ultrasonic input.

3. The system of claim 1, wherein the at least one emitter array is configured to emit an X-ray output and the at least one receiver array is configured to receive an X-ray input.

4. The system of claim 1, wherein the at least one emitter array is configured to emit an NMR output and the at least one receiver array is configured to receive an NMR input.

5. The system of claim 1, wherein the drive unit is configured to drive the at least one emitter array to emit an output of varying phase.

6. The system of claim 5, wherein the acquisition unit is configured to store the input as a three-dimensional grid and assign sections of the grid based on interpreted density values.

7. The system of claim 1, wherein the path determination unit is operative repeatedly to determine an obstacle-free path.

8. The system of claim 1, wherein the indicator unit is configured to provide a visual indication to the operator.

9. The system of claim 1, wherein the indicator unit is configured to provide an audible indication to the operator.

10. The system of claim 1, wherein the indicator unit is configured to provide a first indication where a location and/or angle of approach of the object is close, preferably within a predetermined limit, to an edge of a window as defined by the obstacle-free path.

11. The system of claim 10, wherein the indicator unit is configured to provide a second indication where a location and/or angle of approach of the object is outside a window as defined by the obstacle-free path.

12. The system of claim 1, wherein the at least one emitter array and the at least one receiver array are implemented in a single array element.

13. The system of claim 1, wherein the at least one emitter array and the at least one receiver array are housed in a single transducer element.

14. The system of claim 1, wherein the object comprises a needle, the region of interest comprises an epidural cavity and the structures comprise bone structures.

15. A system for guiding an object, in particular an elongate object, into a region of interest which is reached by maneuvering between structures, the system comprising:
at least one emitter array for emitting an output;
at least one receiver array for receiving an input;
a drive unit operably connected to the at least one emitter array to drive the same to emit an output;
an acquisition unit operably connected to the at least one receiver array to acquire an input as received thereby;
a modeling unit for modeling the input as acquired by the acquisition unit to identify structures as obstacles to guiding of the object; and
a path determination unit for determining an obstacle-free path between the identified obstacle structures in accordance with which the object is in use guided.

16. The system of claim 15, wherein the at least one emitter array is configured to emit an ultrasonic output and the at least one receiver array is configured to receive an ultrasonic input.

17. The system of claim 15, wherein the at least one emitter array is configured to emit an X-ray output and the at least one receiver array is configured to receive an X-ray input.

18. The system of claim 15, wherein the at least one emitter array is configured to emit an NMR output and the at least one receiver array is configured to receive an NMR input.

19. The system of claim 15, wherein the drive unit is configured to drive the at least one emitter array to emit an output of varying phase.

20. The system of claim 19, wherein the acquisition unit is configured to store the input as a three-dimensional grid and assign sections of the grid based on interpreted density values.

21. The system of claim 15, wherein the path determination unit is operative repeatedly to determine an obstacle-free path.

22. The system of claim 15, wherein the at least one emitter array and the at least one receiver array are implemented in a single array element.

23. The system of claim 15, wherein the at least one emitter array and the at least one receiver array are housed in a single transducer element.

24. The system of claim 15, wherein the object comprises a needle, the region of interest comprises an epidural cavity and the structures comprise bone structures.

25. A transducer element comprising a body element which is to be located on a surface of a body, a projecting element which extends from the body element and is configured in use to pierce the body and be located therewithin, and at least one transducer array which is disposed in the projecting element.

26. The transducer element of claim 25, wherein the at least one transducer array comprises at least one emitter array and at least one receiver array.

27. The transducer element of claim 25, wherein the transducer array comprises at least one emitter array.

28. The transducer element of claim 25, wherein the transducer array comprises at least one receiver array.

29. A coupling assembly for movably coupling a device, which includes a body and an elongate object extending therefrom, to a mounting pad, the coupling assembly comprising a connector to which the body of the device is connected and a flexible attachment member which connects the connector to the mounting pad such as to allow for movement of the device relative to the mounting pad.

30. The coupling assembly of claim 29, wherein the attachment member is configured to facilitate controlled movement of the device both axially and laterally relative to the mounting pad.

31. The coupling assembly of claim 30, wherein the connector is fixedly connected to the body of the device.

32. The coupling assembly of claim 31, wherein the connector is slideably connected to the body of the device.

33. The coupling assembly of claim 30, wherein the attachment member comprises a bellows structure.

34. The coupling assembly of claim 29, wherein the mounting pad is for attachment to a body, such as by a glue or an adhesive tape.

35. The coupling assembly of claim 29, wherein the mounting pad comprises a rigid body.

36. The coupling assembly of claim 29, wherein the mounting pad comprises a semi-rigid body.

37. The coupling assembly of claim 29, wherein the mounting pad comprises a flexible body.

38. The coupling assembly of claim 29, further comprising a transducer assembly which comprises the mounting pad and at least one transducer array mounted thereto.

39. The coupling assembly of claim 38, wherein the transducer assembly comprises a plurality of transducer arrays.

40. A transducer assembly for attachment to a surface of a body, the transducer assembly comprising a mounting pad which includes an aperture through which an object is insertable, and at least one transducer array.

41. The tranducer assembly of claim 40, comprising a plurality of transducer arrays.

42. The transducer assembly of claim 41, wherein the aperture comprises an elongate slot and the transducer arrays are disposed to opposed sides of the slot.

43. The transducer assembly of claim 40, wherein the mounting pad is for attachment to the body by a glue or an adhesive tape.

44. The transducer assembly of claim 40, wherein the mounting pad is for attachment to the body by a vacuum.

45. The transducer assembly of claim 40, wherein the mounting pad comprises a rigid body.

46. The transducer assembly of claim 40, wherein the mounting pad comprises a semi-rigid body.

47. The transducer assembly of claim 40, wherein the mounting pad comprises a flexible body.

48. The transducer assembly of claim 47, wherein the mounting pad comprises a tape.

49. An elongate element for insertion into a body, the element incorporating at least one transducer array.

50. The element of claim 49, wherein the element incorporates at least one emitter array.

51. The element of claim 49, wherein the element incorporates at least one receiver array.

52. The element of claim 49, wherein the element incorporates a plurality of transducer arrays.

53. The element of claim 52, wherein the element incorporates at least one emitter array and at least one receiver array.

54. The element of claim 49, wherein the at least one transducer array is located at a tip region of the element.

55. The element of claim 49, wherein the element comprises a tubular element, in particular a needle.

56. An elongate element for insertion into a body, the element incorporating at least one transponder.

57. The element of claim 56, wherein the at least one transponder is located at a tip region of the element.

58. The element of claim 56, wherein the element comprises a tubular element, in particular a needle.

59. A method for use in guiding an object, in particular an elongate object, into a region of interest which is reached by maneuvering between structures, the method comprising the steps of:
emitting an output to the region of interest;
receiving an input from the region of interest;
acquiring the input as received;
modeling the acquired input to identify structures as obstacles to guiding of the object;
determining an obstacle-free path between the identified obstacle structures; and
indicating to an operator whether the object is being guided in accordance with the obstacle-free path.

60. The method of claim 59, wherein the output is an ultrasonic output and the input is an ultrasonic input.

61. The method of claim 59, wherein the output is an X-ray output and the input is an X-ray input.

62. The method of claim 59, wherein the output is an NMR output and the input is an NMR input.

63. The method of claim 59, wherein the output has varying phase.

64. The method of claim 63, wherein the acquiring step comprises the steps of:
storing the input as a three-dimensional grid; and
assigning sections of the grid based on interpreted density values.

65. The method of claim 59, wherein determining step comprises the step of: repeatedly determining an obstacle-free path between the identified obstacle structures.

66. The method of claim 59, wherein the indicating step comprises the step of:
visually indicating to an operator whether the object is being guided in accordance with the obstacle-free path.

67. The method of claim 59, wherein the indicating step comprises the step of:
audibly indicating to an operator whether the object is being guided in accordance with the obstacle-free path.

68. The method of claim 59, wherein the indicating step comprises the steps of:
providing a first indication where a location and/or angle of approach of the object is close, preferably within a predetermined limit, to an edge of a window as defined by the obstacle-free path.

69. The method of claim 68, wherein the indicating step comprises the step of:
providing a second indication where a location and/or angle of approach of the object is outside a window as defined by the obstacle-free path.

70. The method of claim 59, wherein the at least one emitter array and the at least one receiver array are implemented in a single array element.

71. A method of guiding an object, in particular an elongate object, into a region of interest which is reached by maneuvering between structures, the method comprising the steps of:
emitting an output to the region of interest;
receiving an input from the region of interest;
acquiring the input as received;
modeling the acquired input to identify structures as obstacles to guiding of the object;
determining an obstacle-free path between the identified obstacle structures; and
guiding the object in accordance with the obstacle-free path.

72. The method of claim 71, wherein the output is an ultrasonic output and the input is an ultrasonic input.

73. The method of claim 71, wherein the output is an X-ray output and the input is an X-ray input.

74. The method of claim 71, wherein the output is an NMR output and the input is an NMR input.

75. The method of claim 71, wherein the output has varying phase.

76. The method of claim 75, wherein the acquiring step comprises the steps of:
storing the input as a three-dimensional grid; and
assigning sections of the grid based on interpreted density values.

77. The method of claim 71, wherein determining step comprises the step of:
repeatedly determining an obstacle-free path between the identified obstacle structures.

78. The method of claim 71, wherein the guiding step comprises the steps of:
an operator guiding the object in accordance with the obstacle-free path; and indicating to the operator whether the object is being guided in accordance with the obstacle-free path, such as to enable the operator to guide the object in accordance with the obstacle-free path.

79. The method of claim 78, wherein the indicating step comprises the step of:
providing a first indication where a location and/or angle of approach of the object is close, preferably within a predetermined limit, to an edge of a window as defined by the obstacle-free path.

80. The method of claim 79, wherein the indicating step comprises the step of:
providing a second indication where a location and/or angle of approach of the object is outside a window as defined by the obstacle-free path.

81. The method of claim 71, wherein the guiding step comprises the step of:
automatically guiding the object in accordance with the obstacle-free path.

82. The method of claim 71, wherein the at least one emitter array and the at least one receiver array are implemented in a single array element.
